(19) United States
(12) Patent Application Publication
(10) Pub. No.: US 2026/0091241 A1
(43) Pub. Date: Apr. 2, 2026

(54) Automatic Pairing of Electrode Assemblies That Are Used to Apply Tumor Treating Fields (TTFields) to a Subject's Body

(71) Applicant: Novocure GmbH, Baar (CH)

(72) Inventor: Kathryn SMITH-PETERSEN, Carlisle, MA (US)

(73) Assignee: Novocure GmbH, Baar (CH)

(21) Appl. No.: 19/341,399

(22) Filed: Sep. 26, 2025

Related U.S. Application Data
(60) Provisional application No. 63/700,885, filed on Sep. 30, 2024.

(51) Int. Cl. A61N 1/40; A61N 1/04; A61N 1/36

(52) U.S. Cl. CPC A61N 1/40; A61N 1/0476; A61N 1/0496; A61N 1/36002; A61N 1/36034

(57) ABSTRACT

Alternating electric fields (e.g., TTFields) can be imposed in a subject's body using a set of electrode assemblies. The system dynamically configures how alternating voltages are applied to the electrode assemblies by receiving data (e.g., from inertial sensors) associated with the electrode assemblies. Based on this received data, the system determines the relative locations of the electrode assemblies. The system then dynamically configures how alternating voltages are applied to the electrode assemblies based on the determined relative locations. In some embodiments, the dynamic configuration comprises (a) recognizing which of the electrode assemblies are disposed on opposite sides of a region of interest based on the determined relative locations, and (b) grouping the electrode assemblies into pairs so that the alternating voltages will be applied to electrode assemblies that are disposed on opposite sides of the region of interest.

### CROSS REFERENCE TO RELATED APPLICATIONS

This Application claims the benefit of U.S. Provisional Application 63/700,885, filed Sep. 30, 2024, which is incorporated herein by reference in its entirety.

### BACKGROUND

Tumor Treating Fields (TTFields) therapy is a proven approach for treating tumors using alternating electric fields at frequencies e.g., between 50 kHz-5 MHz, more commonly 100-500 KHz. The prior art Optune® system delivers TTFields to patients via four electrode assemblies (a.k.a. transducer arrays) that are placed on the patient's skin near the tumor. These electrode assemblies are arranged in two pairs, e.g., with one pair of electrode assemblies positioned to the left and right of the tumor, and the other pair of electrode assemblies positioned anterior and posterior to the tumor. Each electrode assembly is connected via a multi-wire cable to an AC signal generator. Optune's AC signal generator (a) sends an AC current through the anterior/posterior (A/P) pair of electrode assemblies for 1 second, which induces an electric field with a first direction through the tumor; then (b) sends an AC current through the left/right (L/R) pair of arrays for 1 second, which induces an electric field with a second direction through the tumor; then repeats steps (a) and (b) for the duration of the treatment.

Optune® will not work properly unless each pair of electrode assemblies are placed on the appropriate locations on a patient's body (e.g., with the L/R electrode assemblies positioned on opposite sides of the tumor, and with the A/P electrode assemblies positioned on opposite sides of the tumor). To help ensure that this occurs, the connectors on the electrode assemblies are color-coded (i.e., black for the A/P electrode assemblies, and white for the L/R electrode assemblies) so that they will only mate with the corresponding connector on the AC signal generator.

### SUMMARY OF THE INVENTION

One aspect of the invention is directed to a first method for using a plurality of electrode assemblies positioned on or in a subject's body to apply alternating electric fields to a region of interest within the subject's body. The first method comprises receiving data corresponding to at least some of the plurality of electrode assemblies, determining relative locations of at least some of the plurality of electrode assemblies based on the received data, and dynamically configuring how alternating voltages are applied to the plurality of electrode assemblies based on the determined relative locations. The alternating voltages have frequencies between 50 kHz and 1 MHz.

In some instances of the first method, the dynamically configuring comprises recognizing which of the plurality of electrode assemblies are disposed on opposite sides of the region of interest based on the determined relative locations, and grouping the electrode assemblies into pairs so that the alternating voltages will be applied to electrode assemblies that are disposed on opposite sides of the region of interest.

In some instances of the first method, the dynamically configuring comprises recognizing which of the plurality of electrode assemblies are disposed on opposite sides of the region of interest based on the determined relative locations, and grouping the electrode assemblies into groups so that the alternating voltages will be applied to electrode assemblies that are disposed on opposite sides of the region of interest.

In some instances of the first method, each of the plurality of electrode assemblies is connected to a respective one of a plurality of output terminals of a switch array, and the dynamically configuring comprises dynamically configuring electrical connections between output terminals of an AC signal generator and the output terminals of the switch array.

In some instances of the first method, the determining of relative locations comprises determining relative locations for a subset of the plurality of electrode assemblies comprising fewer electrode assemblies than a total number of the plurality of electrode assemblies.

In some instances of the first method, the receiving of data comprises receiving at least one signal from each of a plurality of location identifying elements, each of the plurality of location identifying elements is associated with a given one of the plurality of electrode assemblies positioned on the subject's body, and the determining of relative locations is based on the at least one signal received from the each of the plurality of location identifying elements.

Optionally, in the instances described in the previous paragraph, the receiving of data occurs in response to at least one issued prompt, and the method further comprises issuing the at least one prompt.

Optionally, in the instances described in the previous paragraph, each of the location identifying elements comprises an inertial sensor. Optionally, in these instances, each of the inertial sensors comprises at least one of an accelerometer, a gyroscope, and a magnetometer.

In some instances of the first method, the receiving of data comprises receiving at least one signal from each of a plurality of location identifying elements, each of the plurality of location identifying elements is associated with a given one of the plurality of electrode assemblies positioned on the subject's body, the determining of relative locations is based on the at least one signal received from the each of the plurality of location identifying elements, and each of the location identifying elements comprises an optical sensor.

In some instances of the first method, the receiving of data comprises receiving at least one signal from each of a plurality of location identifying elements, and each of the plurality of location identifying elements is associated with a given one of the plurality of electrode assemblies positioned on the subject's body. The determining of relative locations is based on the at least one signal received from the each of the plurality of location identifying elements, and each of the location identifying elements comprises a switch that is actuatable by the subject. Optionally, in these instances, the receiving of data comprises receiving an indication that each of the switches has been actuated by the subject at a respective time after a corresponding prompt has been issued to the subject.

In some instances of the first method, the receiving of data comprises receiving captured image data of the plurality of electrode assemblies positioned on the subject's body. And the determining of relative locations comprises (a) identifying, using a trained machine learning system implementing an electrode assembly recognition model, the at least some of the plurality of electrode assemblies included in the image data, and (b) determining relative locations for the at least some of the plurality of electrode assemblies based on the identified electrode assemblies included in the image data.

Another aspect of the invention is directed to a first apparatus that comprises a controller, an AC signal generator, and a switch array. The AC signal generator generates an output at a frequency between 50 kHz and 1 MHz. The switch array is electrically coupled to the AC signal generator, and is dynamically configurable to route the output of the AC signal generator to selected ones of a plurality of electrode assemblies based on at least one control signal that arrives from the controller. The controller is configured to select which of the plurality of electrode assemblies will receive the output of the AC signal generator at respective times based on signals that arrive from a plurality of location identifying elements, each of which is associated with a given one of the plurality of electrode assemblies.

In some embodiments of the first apparatus, the controller is configured to (a) recognize which of the plurality of electrode assemblies are disposed on opposite sides of the region of interest based on the signals that arrive from the plurality of location identifying elements, and (b) group the electrode assemblies into pairs so that the output of the AC signal generator is routed to electrode assemblies that are disposed on opposite sides of the region of interest.

In some embodiments of the first apparatus, the controller is configured to (a) recognize which of the plurality of electrode assemblies are disposed on opposite sides of the region of interest based on the signals that arrive from the plurality of location identifying elements, and (b) group the electrode assemblies into groups so that the output of the AC signal generator is routed to electrode assemblies that are disposed on opposite sides of the region of interest.

In some embodiments of the first apparatus, the controller is configured to cause, based on the signals that arrive from the plurality of location identifying elements, actuation of one or more switches of the switch array to electrically connect the output of the AC generator to pairs of output ports of the switch array. Optionally, in these embodiments, the controller is further configured to cause, based on the signals that arrive from the plurality of location identifying elements and based on a schedule specifying the timing at which different pairs of electrode assemblies should be activated or de-activated, actuation of one or more switches of the switch array to electrically connect the output of the AC generator to the pairs of output ports of the switch array.

Some embodiments of the first apparatus further comprise the plurality of electrode assemblies and the plurality of location identifying elements. Optionally, in these embodiments, each of the electrode assemblies is configured to adhere to the subject's body, and each of the location identifying elements is configured to produce at least one signal representative of a relative location of the associated given one of the plurality of electrode assemblies, and to communicate the at least one signal to the controller.

Another aspect of the invention is directed to a first electrode assembly for applying alternating electric fields to a region of interest within a subject's body. The electrode assembly comprises one or more conductive pads, a layer of conductive adhesive, an inertial sensor, and a cable. Each of the conductive pads has a front surface, and a collective area of the one or more conductive pads is at least 5 cm². The layer of conductive adhesive is disposed on the front surface of the one or more conductive pads. The inertial sensor is configured to generate an output. And the cable is configured to route a signal from an AC signal generator to the one or more conductive pads.

In some embodiments of the first electrode assembly, the cable is further configured to route the output of the inertial sensor to a remote controller.

Some embodiments of the first electrode assembly further comprise a communication module configured to wirelessly transmit the output of the inertial sensor to a remote controller.

### BRIEF DESCRIPTION OF THE DRAWINGS

FIG. 1 is a schematic diagram of an example system to determine the relative locations of electrode assemblies positioned on a subject's body, and to perform TTFields therapy according to the determined relative locations.

FIG. 2 is a schematic diagram of another example system to determine the relative locations of electrode assemblies using a machine learning approach, and to perform TTFields therapy according to the determined relative locations.

FIG. 3 is a flowchart of an example procedure for using a plurality of electrode assemblies positioned on or in a subject's body to apply alternating electric fields to a region of interest within the subject's body.

Various embodiments are described in detail below with reference to the accompanying drawings, wherein like reference numerals represent like elements.

### DESCRIPTION OF THE PREFERRED EMBODIMENTS

Although the prior art approach of color coding the connectors on the electrode assemblies is workable, problems can arise if a subject ignores the color coding and positions the prior art electrode assemblies improperly. Assume, for example, that a subject (1) positions the two electrode assemblies with black connectors on the anterior and left sides of their head, (2) positions the two electrode assemblies with white connectors on the posterior and right sides of their head, and (3) subsequently plugs the white and black connectors into the correspondingly-colored ports of the AC signal generator. This scenario will cause the system to apply AC voltages (a) between the anterior and left electrode assemblies, and (b) between the posterior and right electrode assemblies. The TTFields that are generated using this setup will typically be relatively weak in the middle section of the subject's head, with an associated reduction in the efficacy of treatment. In addition, because the AC voltages are being applied between neighboring electrode assemblies (rather than opposite electrode assemblies), the adjacent edges of the electrode assemblies will begin to heat up, which will cause the prior art system to reduce its output current to prevent overheating. And this reduction in current will further limit the efficacy of treatment.

Furthermore, because all four of the electrode assemblies are not identical, they are non-interchangeable, and must be provided to the subject in sets. And the non-interchangeable nature of the electrode assemblies requires the subject to take more care when applying each electrode assembly to respective locations on their body.

With the methods and embodiments described herein, the system can determine the relative locations of the electrode assemblies after they have been positioned on the subject's body, and dynamically configure how alternating voltages are applied to those electrode assemblies based on the determined relative locations. These methods and embodiments advantageously ensure that the electrode assemblies will never be improperly positioned or improperly connected to the AC signal generator. They also advantageously allow all four electrode assemblies to be interchangeable, which means that the subject does not have to be so careful when they position the electrode assemblies on their body.

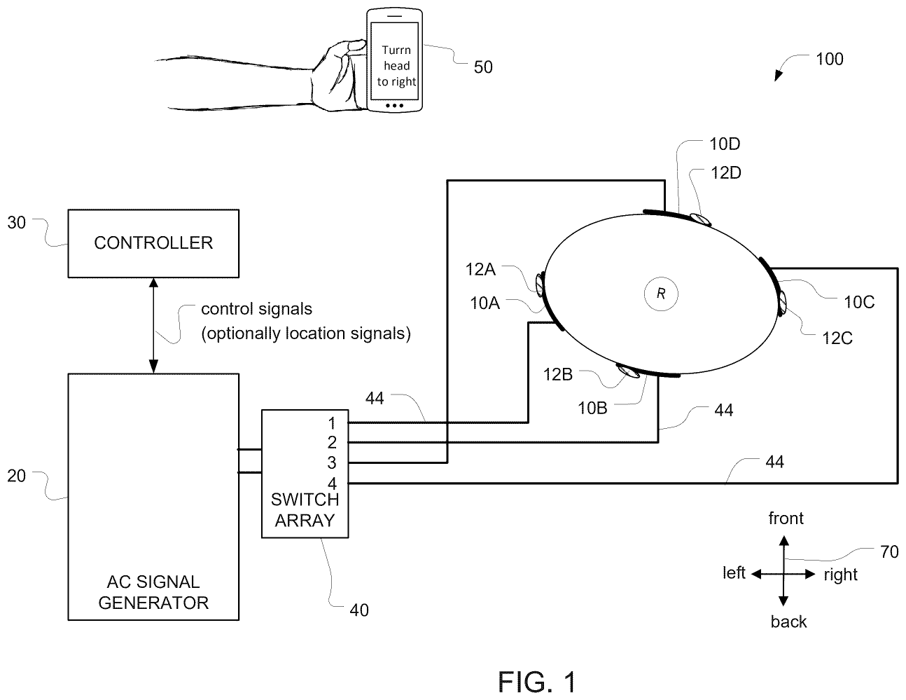

FIG. 1 is a schematic diagram of a system 100 for performing TTFields therapy in a portion of the subject's body that includes at least one region of interest. FIG. 1 includes a top view of a representation of subject's body part (namely, the subject's head) in which a tumor is located in the region of interest R. To treat the tumor in the region of interest, four electrode assemblies 10 are placed around the region of interest R. While FIG. 1 illustrates four electrode assemblies, a different number of electrode assemblies (e.g., 3-10) may be deployed on the subject's body, as may be therapeutically necessary or desired.

A wide variety of electrode assemblies can be used in the implementations described herein, including but not limited to the electrode assemblies described in US 2023/0043071, entitled “Electrode Assembly for Applying Tumor Treating Fields (TTFields) that Include a Sheet of Graphite,” US 2021/0402179, entitled “Flexible Transducer Arrays with a Polymer Insulating Layer for Applying Tumor Treating Fields (TTFields),” and U.S. Pat. No. 8,715,203, entitled “Composite Electrode.” Each of these documents is incorporated herein by reference in its entirety. The electrode assemblies can all be identical or can be different, based on the specific needs of a particular situation.

The electrode assemblies 10 can be adhesively secured to the skin, e.g., using a conductive adhesive. For example, the electrode assemblies can be secured using an adhesive layer disposed on the contact surface of the electrode assemblies (i.e., the surfaces of the electrode assemblies facing the subject's skin). The electrode assemblies can be secured to the subject's body (to maintain their positions) using other electrode-securing mechanisms, e.g., similar to bandages in which the front surface is at least partly covered with an adhesive that adheres to the subject's skin.

In the embodiment depicted in FIG. 1, each electrode assembly 10 is equipped with a corresponding location identifying element 12. This facilitates the automatic configuration procedure described herein, in which the relative locations of each of the electrode assemblies is determined, along with their associated connectivity to an output port (e.g., via cables 44). The location identifying elements 12 may be mounted on, or in, their respective electrode assemblies. Although FIG. 1 schematically depicts the location identifying elements as being mounted on the respective electrode assembly structures, one or more of the location identifying elements 12 may be disposed within the structure constituting the respective electrode assembly. Note that the electrode assemblies 10 and the location identifying elements 12 are respectively labeled 10A-D and 12A-D in FIG. 1 so that then can be differentiated from each other in the discussion that follows.

Each of the location identifying elements 12 sends at least one signal to a controller 30. The controller 30 receives these signals and determines the relative locations of the electrode assemblies 10 based on these signals. As will be discussed in greater detail below, in addition to being configured to determine the relative locations based on signals it receives from the various location identifying elements, the controller 30 (e.g., a processor-based controller) is configured to control the operation of an AC signal generator 20 (and, in some embodiments, to dynamically control electrical connectivity configurations of electrical paths that can be formed in a switch array 40) based on the determined relative locations. In some examples, the signals from the location identifying elements 12 may be communicated to some other dedicated device (or even to one or more of the location identifying elements) to determine the relative locations for the electrode assemblies.

Various types of location identifying elements can be used to communicate signals from which the relative locations can be determined (at the controller 30 or elsewhere).

In some embodiments the location identifying elements 12 are inertial sensors such as, for example, an accelerometer, a gyroscope, and/or a magnetometer. The motion of the electrode assembly is determined based on change in the electrode assembly's orientation as detected and measured through the one or more inertial (orientation) sensors disposed on or in a respective electrode assembly. An accelerometer, for example, is an instrument that detects linear motion, e.g., translation in a plane, such as a local horizontal plane, measured with reference to at least two axes. An accelerometer can thus be used to also measure an object's tilt or rotation in a 3-D space (such as a Cartesian coordinate space). In some embodiments, an accelerometer can be an arrangement of three 1-D accelerometers that together form a 3D accelerometer. An accelerometer can be implemented, in some embodiments, using micro-electro-mechanical-system (MEMS) technology. A gyroscope sensor, which can also be implemented based on MEMS technology, is configured to sense motion about, for example, three orthogonal axes. Gyroscope sensors can be implemented as a single-axis gyroscope, a double-axis gyroscope, or a 3-D gyroscope. A magnetometer is configured to measure a magnetic field intensity and/or direction, and may, in some embodiments, measure absolute orientation with respect to the magnetic north (which can be converted to orientation with respect to true north). A MEMS-based magnetometer may be configured to detect motion caused by the Lorentz force produced by a current through a MEMS conductor. Other types of magnetometers include Hall effect magnetometers, rotating coil magnetometers, etc.

In these embodiments, measurements performed by the inertial sensor(s) of each electrode assembly are used to generate at least one signal encoding data representative of detected motion of that electrode assembly. The at least one signal is then communicated to the controller 30 (or to some other processing device in communication with the controller 30). The processing of the inertial sensor(s) measurements can be e.g., performed by a communication module (not shown) that can be part of the inertial sensor, or part of the respective electrode assembly. The communication module can include a signal modulation unit (not shown) to generate an electrical or electromagnetic signal for transmission to the controller 30 through a cable (such as the cable 44) to the controller via the AC generator 20 (and the switch array 40, if used). Alternatively, the at least one signal generated can be transmitted wirelessly to the controller 30 via a transceiver included with the communication module of the inertial sensor or with the respective electrode assembly and received by a transceiver provided with the controller 30. Note that wireless communication encompasses all types of wireless technologies and protocols, including wireless local access network (WLAN) technologies, which may be based on WiFi type implementations, wireless wide area networks (WWAN) technologies, which may be established using cellular links (e.g., according to 4G, 5G, 6G, and future generation technologies), short range wireless technologies (e.g., Bluetooth), etc.

The at least one signal communicated by each inertial sensor 12 can include data indicating directions (e.g., in a 3D space) at which a respective electrode assembly 10 is moving or has moved (the data represented by the at least one signal may also include an identifier to uniquely identify the particular electrode assembly 10 associated with the measuring inertial sensor 12). For example, when the electrode assemblies and their associated inertial sensors (accelerometers in this example) are positioned on the subject's head, rotation of the subject's head will cause all of the electrode assemblies 10 to move in different directions. Thus, referring to the frame of reference 70, when the subject's head is turned to the right, the electrode assembly 10C (and its corresponding location identifying element 12C) will move in a backward and leftward directions, while the opposite electrode assembly 10A (and its corresponding location identifying element 12A) will move in a general frontward and rightward direction. On the other hand, a nodding motion of the subject's head will produce a different combination of movements of the various location identifying elements 12. The directions of motion of the electrode assemblies can therefore be used to determine their relative location to each other (e.g., electrode assembly 10A is located opposite electrode assembly 10C). By also knowing the general direction that the subject is facing, the relative locations of the various assemblies can be determined and used to dynamically configure the electrical connectivity of the AC output ports of the switch array 40 to the various electrode assemblies 10.

In some embodiments, the determination of the relative locations of the electrode assemblies 10 can be implemented fully automatically. Assume, for example, that four identical electrode assemblies 10 (each of which includes a respective accelerometer 12) are positioned on the subject's head, and each of the electrode assemblies 10 has a respective cable 44 that is plugged into the switch array 40 (as depicted in FIG. 1). If the signals from the accelerometer 12 in each of the electrode assemblies 10 are routed (e.g., via the switch array 40 and the AC signal generator 20) to the controller 30, the controller 30 will have access to the output signals from all the accelerometers 12. And by analyzing these output signals and recognizing specific patterns in those signals (based on knowledge of what movements are possible for a human body), the controller 30 can determine which electrode assemblies 10 are located on opposite sides of the region of interest (ROI).

For example, if four electrode assemblies 10 are mounted on the front, back, left, and right sides of the subject's head, and the subject nods their head, the front and back accelerometers 12 will both detect up and down movements that are out of phase with each other, while the left and right accelerometers 12 will detect in-phase rotational movements.

And if the subject turns their head from left to right, all four accelerometers 12 will detect rotational movements that are all in phase. Thus, if the controller 30 recognizes these patterns (at different times while the subject is wearing the electrode assemblies 10), it can conclude that the two accelerometers 12 that are detecting out-of-phase up and down movements are located on opposing electrode assemblies 10, and assign those two electrode assemblies 10 to a first pair. The remaining two electrode assemblies 10 are then assigned to a second pair.

The controller 30 then configures the switch array 40 so that AC voltages are applied between the electrode assemblies 10 within any given pair, as described below.

In other embodiments, the determination of which electrode assemblies 10 are located on opposite sides of the ROI can be implemented fully automatically by a machine learning (ML) system that is trained to recognize patterns that exist between the outputs of the various accelerometers 12 in view of the anatomy of the subject's body. In this example, the ML system inputs the signals from all four accelerometers 12 over a period of time (e.g., at least 10 s, at least 30 s, or at least 1 minute), and determines which electrode assemblies 10 should be paired up with each other. The controller 30 then configures the switch array 40 so that AC voltages are applied between the electrode assemblies 10 within any given pair, as described below.

In other embodiments, the determination of the relative locations of the electrode assemblies 10 is semi-automatic and relies on prompting the subject (e.g., using a smartphone 50) in communication (e.g., wireless communication) with a device (e.g., the controller 30) that performs the initial configuration procedure that includes location determination of the electrode assemblies. In the example of FIG. 1, the smartphone 50 receives and presents a prompt directing the subject to turn his/her head to the right. (The prompts may be pre-programmed in advance or downloaded). In response to receipt of the prompt, the subject turns their head to the right, causing movement of the electrode assembly 10D to the right (as measured by its inertial sensor 12D), which indicates that the subject is facing the front direction. Determination of the direction the subject is facing, together with the directions of motions measured by at least some of the inertial sensors 12, allows determination of the relative (or even absolute) locations of the various electrode assemblies 10 associated with the inertial sensors 12. The controller 30 then configures the switch array 40 so that AC voltages are applied between the electrode assemblies 10 within any given pair, as described below.

In other embodiments, the location identifying elements 12 can be implemented using user-actuatable switches that transmit an electrical signal when pressed (in this case to the controller 30 of FIG. 1). In order to properly determine relative locations of the switches, and thus the relative locations of their associated electrode assemblies 10, the switches are pressed in some particular order based on prompts delivered to the subject/technician. Here too, the prompts may be delivered using the smartphone device 50.

In the switch-based embodiments, the subject/technician receives prompts to actuate the switches in a particular pattern. For example, the subject could be prompted to first press the switch that is positioned on the electrode assembly that is located closest to the subject's nose. In the example of FIG. 1, the electrode assembly closest to the subject's nose is electrode assembly 10D, and accordingly the subject or the subject/technician actuates the switch of the location identifying element 12D. Upon pressing the switch of location identifying element 12D, at least one signal is transmitted either wirelessly to the controller 30, or via a wired path that extends from the electrode assembly 10D, through the cable 44 connecting it to the switch array 40 and the AC signal generator 20, to the controller 30. Note that when transmitting a signal through the wired path, the AC signal generator 20 and the switch array 40 may need to be configured to be in a state where there is a complete electrical path between all the ports of the switch array 40, the AC signal generator 20, and the controller 30. Upon receipt of the at least one signal transmitted by the location identifying element 12D, the prompting device prompts (optionally in response to a signal sent from the controller 30, confirming receipt of the signal from the location identifying element 12D) to generate a subsequent prompt instructing the subject or technician to actuate the next switch located on the next electrode assembly placed on the subject's head. This procedure is then repeated for the remaining electrode assemblies 10.

In other embodiments, the location identifying elements 12 can be implemented using optical sensors. Such optical sensors can be configured to detect optical signals (optionally at some pre-specified wavelengths), and can thus transmit to the controller 30 a signal indicating detection of the optical signal and measurements associated with the detected signal. In some examples, a technician or a subject may be prompted to shine a light (e.g., from a pointer device, a flashlight such as one that can be activated on a wireless phone device, etc.) on an optical sensor of a first electrode assembly 10 (e.g., the electrode assembly closest to the subject's nose). Subsequently, the technician or the subject are prompted to proceed in sequence to shine the light on optical sensors of neighboring electrode assemblies 10. In some examples, a light source located at a known position away from the subject can shine a light detected by some or all of the optical sensors. The optical sensors, in this example, measure the light intensity of the detected light, which in turn can be used to determine the relative locations of the electrode assemblies to each other. Other ways in which light detected by optical sensors to determine relative locations of the electrode assemblies 10 can also be used. Upon detecting optical signals, the sensors cause signals, encoded with data indicating the optical measurements (and optionally the identities of the electrode assemblies associated with those measurements), to be transmitted (wirelessly, or through a wired path passing through the switch array 40 and the AC signal generator 20) to the controller 30.

In addition to the inertial sensors, actuatable switches, and optical sensors described herein, other, different, types of location identifying elements 12 may be used to facilitate determination of locations of electrode assemblies 10 associated with such location identifying elements.

Regardless of which approach is used to determine the relative locations of the electrode assemblies 10, it may not be necessary to actively determine the relative locations of each and every one of the electrode assemblies. Instead, it may be possible to determine the relative locations for a subset of the plurality of electrode assemblies 10, and subsequently infer the relative locations for the remaining electrode assemblies. Assume, for example, a situation in which there are four electrode assemblies 10, and based on any of the approaches described above, the controller 30 has determined that a first one of the electrode assemblies 10 is positioned on the right side of the subject's head and a second one of the electrode assemblies 10 is positioned on the left side of the subject's head. Because those two electrode assemblies 10 are positioned on opposite sides of the region of interest, the controller 30 can group those two electrode assemblies 10 into a first pair, and group the remaining two electrode assemblies 10 into a second pair (based on an inference that the remaining two electrode assemblies 10 must be opposite to each other).

As noted, signals transmitted by the location identifying elements 12 are received by, for example, the controller 30, which uses the data encoded on the signals to determine relative locations of at least some of the plurality of electrode assemblies 10. The controller 30 then uses the determined relative locations of the electrode assemblies to dynamically configure how alternating voltages are applied to opposing pairs of electrode assemblies 10. More specifically, the controller 30 is configured to select, based on the relative locations of the electrode assemblies 10 (e.g., as determined based on the signals received from the location identifying elements 12), which of the plurality of electrode assemblies 10 will receive the output of the AC signal generator 20 at respective times.

The controller 30 can dynamically configure the system by recognizing which of the plurality of electrode assemblies 10 are disposed on opposite sides of the ROI based on the determined relative locations. The controller 30 then groups the electrode assemblies 10 into pairs so that the alternating voltages will be applied to electrode assemblies that are disposed on opposite sides of the ROI.

Assume, in a first example, that a user with a brain tumor has positioned a first electrode assembly 10 on the right side of their head, a second electrode assembly 10 on the left side of their head, a third electrode assembly 10 on the front of their head, and a fourth electrode assembly 10 on the back of their head. Based on the determined relative locations, the controller 30 recognizes that the first and second electrode assemblies are disposed on opposite sides of the ROI, and that the third and fourth electrode assemblies are disposed on opposite sides of the ROI. The controller 30 will then dynamically configure how the alternating voltages are applied to those pairs of electrode assemblies. A suitable pattern for applying alternating voltages to the electrode assemblies in this example would be to (a) apply the alternating voltages across the pair that includes the first and second electrode assemblies for a period of time (e.g., 0.25-10 s); (b) apply the alternating voltage across the pair that includes the third and fourth electrode assemblies for a similar period of time; and (c) repeat steps (a) and (b) for the duration of the treatment.

Now assume, in a second example, that the same user has positioned the first electrode assembly 10 on the right side of their head, the second electrode assembly 10 on the front side of their head, the third electrode assembly 10 on the left of their head, and the fourth electrode assembly 10 on the back of their head. Based on the determined relative locations, the controller 30 recognizes that the first and third electrode assemblies are disposed on opposite sides of the ROI, and that the second and fourth electrode assemblies are disposed on opposite sides of the ROI. The controller 30 will then dynamically configure how the alternating voltages are applied to those pairs of electrode assemblies. A suitable pattern for applying alternating voltages to the electrode assemblies in this example would be to (a) apply the alternating voltages across the pair that includes the first and third electrode assemblies for a period of time (e.g., 0.25-10 s); (b) apply the alternating voltage across the pair that includes the second and fourth electrode assemblies for a similar period of time; and (c) repeat steps (a) and (b) for the duration of the treatment.

These two examples illustrate how, regardless of which electrode assembly 10 is positioned on which part of the subject's body, the system can dynamically configure the application of the alternating voltages so that alternating voltages are applied between electrode assemblies that are disposed on opposite sides of the region of interest.

Note that the dynamic configuration of the electrode assemblies 10 is not limited to selecting the electrode assemblies so that an alternating voltage is applied between only a single electrode assembly 10 on one side of the ROI and only a single electrode assembly 10 on the opposite side of the ROI at any given moment in time (as described in the examples above). To the contrary, the dynamic configuration can be such that an alternating voltage is applied to the electrode assemblies in other grouping configurations. For example, the dynamic configuration can be such that an alternating voltage is applied between (a) both the left and back electrode assemblies 10 and (b) both the right and front electrode assemblies 10. Alternatively, the dynamic configuration of the electrode assemblies 10 can be such that an alternating voltage is applied between (a) the left electrode assembly and (b) both the right and front electrode assemblies. As yet another alternative, the dynamic configuration of the electrode assemblies can be such that an alternating voltage is applied between (a) the left electrode assembly and (b) both the right and back electrode assemblies. These grouping configurations will yield different electric field strengths in different parts of the subject's head, which can be beneficial e.g., in situations where the ROI is not located in the exact center of the subject's head. The controller 30 can set up any of these grouping configurations by sending signals to the switch array 40 that cause the switch array 40 to route the output of the AC signal generator 20 to the corresponding electrode assemblies.

The controller 30 selects the electrode assemblies to which AC voltage is directed from the AC signal generator by controlling the actuation of one or more switches of the switch array 40 to electrically connect the output of the AC generator to pairs of output ports (such as output ports #1 and #4) of the switch array 40. For example, the controller 30 can cause (through control signals sent to the AC signal generator 20 and/or the switch array 40) to actuate switches, (whether they are semiconductor-based switches or electro-mechanical switches) to form dynamically configurable electrical paths between the output of the AC generator and the output ports of the switch array 40. These electrical paths are maintained for some period of time that depends on a schedule controlling which pairs of electrode assemblies become activated over different time periods. In other words, the controller 30 electrically connects the electrode assemblies 10 to the AC signal generator 20 in a desired temporal order so that different pairs of opposing electrode assemblies 10 become active during specified time periods.

The AC signal generator 20 is configured to controllably generate and apply AC signals (e.g., via the switch array 40) to opposing pairs of the electrode assemblies 10 that are positioned on the subject's body. The AC signal generator 20 is configured to apply an AC voltage with a frequency of between 50 kHz and 1 MHz (e.g., 50-500 kHz, 75-300 kHz, or 150-250 kHz), e.g., at voltage levels of 50-150 VRMS. One way to implement the AC signal generator 20 is described in U.S. Pat. No. 9,910,453, which is incorporated herein by reference in its entirety. Alternatively, a variety of alternative approaches for implementing the AC signal generator 20 that will be apparent to persons skilled in the relevant arts can be used. In some embodiments, the controller 30 also controls the operation of the AC signal generator 20 based on operating temperature measurements (e.g., measured by thermistors positioned on the electrode assemblies) to adjust the AC signal that is applied to the electrode assemblies 10 in order to maintain their temperatures below a safety threshold (e.g., 39° C.).

Control signals provided by the controller 30 cause switches (e.g., semiconductor-based or electromechanical switches) within the switch array 40 to be actuated into different states that allow different electrical paths to be established between the input ports and output ports of the switch array 40. These electrical paths can be established and then removed according to a timing schedule prepared in accordance with the therapeutic needs of the subject, resulting in opposing pairs of electrode assemblies 10 receiving AC voltage at levels and time intervals appropriate for the particular subject. Note that the switch array 40 and the AC generator 20 can be combined into a single device, or can include, as depicted in FIG. 1, separate connectable units.

Thus, an apparatus for applying alternating electric fields to a region of interest within a subject's body includes, in various embodiments, a controller 30, an AC signal generator 20 that generates an AC output at a frequency between 50 kHz and 1 MHz, and a switch array 40 that is electrically coupled to the AC signal generator 20. The switch array 40 is dynamically configurable to route the output of the AC signal generator 20 to selected ones of a plurality of electrode assemblies 10 based on at least one control signal that arrives from the controller 30. The controller 30 is configured to select which of a plurality of electrode assemblies 10 will receive the output of the AC signal generator 20 at respective times based on signals that arrive from a plurality of location identifying elements 12 (each of which is associated with a given one of the plurality of electrode assemblies 10). Note that, in some examples, an apparatus that includes the controller 30, the AC signal generator 20, and the switch array 40 can be provided in a single housing, or can be an arrangement of electrically connected individual units.

In some embodiments, the apparatus can further include the plurality of electrode assemblies 10 and the plurality of location identifying elements 12. Each of the electrode assemblies 10 may be configured to adhere to the subject's body, and each of the location identifying elements 12 may be configured to produce at least one signal representative of a relative location of the associated given one of the plurality of electrode assemblies 10, and to communicate the at least one signal to the controller 30.

In some embodiments (e.g., as depicted in FIG. 1), the system includes a set of electrode assemblies 10, each of which includes (i) one or more conductive pads having a front surface, wherein a collective area of the one or more conductive pads is at least 5 cm², (ii) a layer of conductive adhesive disposed on the front surface of the one or more conductive pads; (iii) an inertial sensor 12; and (iv) a cable 44 configured to route a signal from an AC signal generator 20 to the one or more conductive pads of the electrode assembly 10. The cable 44 may also be configured to route the output of the inertial sensor 12 to a remote controller. The electrode assemblies 10 of this example can further include a communication module configured to wirelessly transmit the output of the inertial sensor 12 to a remote controller (as noted, the communication module can alternatively be disposed in the inertial sensor). Note that location identifying elements other than an inertial sensor may be used with this example electrode assembly.

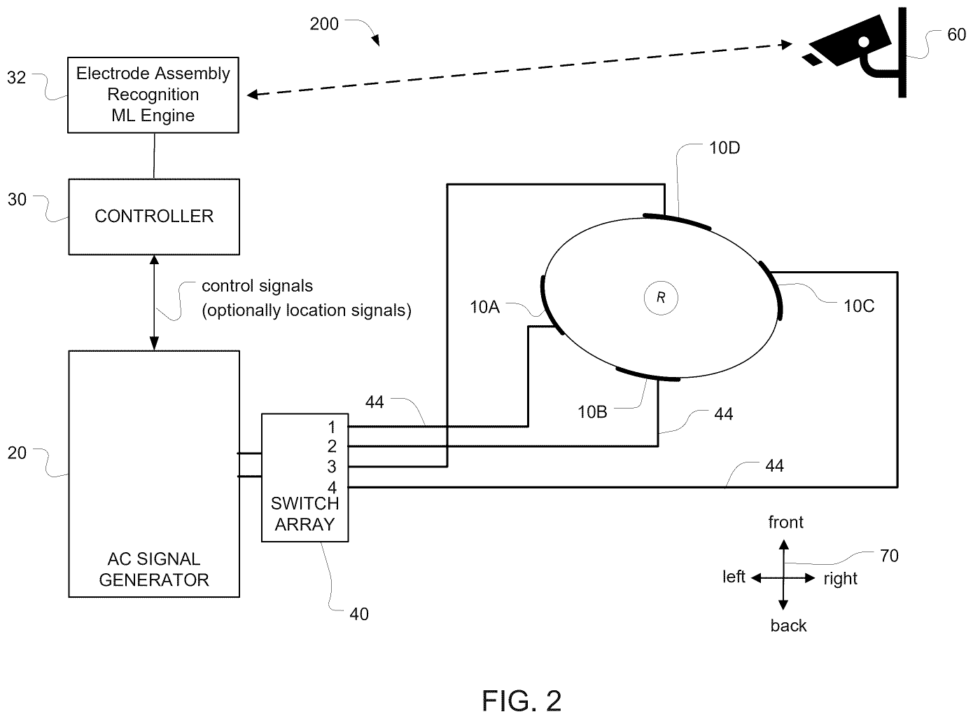

FIG. 2 is a schematic diagram of another example system 200 that is similar to the system 100 except that the location determination is performed using machine learning.

More particularly, the system 200 includes an image capture device 60 (e.g., a digital camera, or a video camera) that can capture an image of a scene that includes the subject, the various deployed electrode assemblies 10 on the subject's body, and optionally the cables 44 extending from the electrode assemblies 10 to the respective ports of the switch array 40.

Once an image of the scene is captured, the image is forwarded, wirelessly or through a wired connection, to a machine learning (ML) engine 32 (which can be implemented as part of the controller 30) that is trained to identify from the image data electrode assemblies (of different types and sizes) and/or electrical cables extending from the assemblies. The ML engine 32 can be implemented using different types of learning architectures, configurations, and/or implementation approaches. Examples of learning machine architectures include neural networks, including convolutional neural network (CNN), feed-forward neural networks, recurrent neural networks (RNN), etc. The output of the ML engine 32 can be a processed image in which labels (text, geometric shapes, or other types of representations) identifying electrode assemblies (and/or the cables) and their relative locations and orientations in the scene are shown. The labels can be overlaid on the original image, or the original image (or at least some of its features) may be suppressed or removed.

Having identified, using the ML engine 32, the various electrode assemblies 10 (and optionally the respective cables 44 extending from them), the controller 30 computes the relative locations of the electrode assemblies appearing in the captured image, and determines the pairing of electrode assemblies (and optionally the ordered schedule of their activation and de-activation). In some situations (e.g., where the captured image of the scene also includes details of the cables and their connections to the output ports of the switch array 40 or the AC signal generator 20), the controller 30 can match the identified electrode assemblies to the AC output ports to which they are connected without requiring the user to perform any actions (e.g., moving their head, pressing a button, etc.). Furthermore, the controller 30 can also derive a timing schedule for activating and de-activating the various electrode assembly pairings.

In other situations (e.g., where the connectivity of the cables 44 to the output ports of the switch array 40 is not visible in the captured image, or there is some ambiguity about which cable 44 is connected to which port), the controller 30 may require additional information to identify which ports the electrode assemblies 10 are connected to. In these embodiments, the controller 30 can communicate instructions or prompts (e.g., to move their head, press a button, etc.) to the user/technician to ascertain which ports of the switch array 40 are connected to which electrode assembly 10.

In operation, and with reference to both FIGS. 1 and 2, a technician or a subject undergoing TTFields therapy places electrode assemblies 10 on the subject's body near or around the region of interest. In the embodiments described herein it is not necessary for specific electrode assemblies, with specific markings or colors or other distinguishing characteristics, to be placed at the various specified positions near or around the region of interest. Rather, selections of which electrode assembly to put at a particular position near or around the region of interest can be arbitrary. Typically, all the electrode assemblies will be of the same type. But this is not required, and in some embodiments different types of electrode assemblies can be mixed together.

Cables 44 extending from the electrode assemblies 10 can next be coupled to AC output ports of either an AC signal generator (such as the AC signal generator 20, if it has enough ports) or to a switch array (such as the switch array 40, whose input ports are electrically coupled to the output of the AC signal generator 20). The cables 44 extending from the electrode assemblies 10 can be connected to arbitrary AC output ports of the AC signal generator 20 or the switch array 40. For example, as shown in FIGS. 1 and 2, electrode 10A is electrically coupled to output port #1 of the switch array 40, while its paired electrode assembly 10C is connected to the output port #4 of the switch array 40.

Having positioned electrode assemblies 10 near or around the region of interest, and having connected those electrode assemblies 10 to output ports of the switch array 40 via cables 44, the particular locations and identity of the various electrode assemblies deployed on the subject's body and which output port they are connected to are determined. In the embodiments illustrated in FIG. 1, a configuration procedure can be performed in which location identifying elements 12 are used to communicate signals (wirelessly or through the cables attaching the electrode assemblies to the output ports) to a controller 30 that determines, based on the signals, the relative locations of the electrode assemblies 10, and optionally the output port to which the assemblies are connected. As described above, the location identifying elements can be, for example, inertial sensors that detect and measure directions of motion of the location identifying elements, optical sensors that can detect and measure light levels from a light source(s), user-actuatable mechanical switches, and so on.

When the location identifying elements 12 include inertial sensors, the configuration procedure can include providing prompts instructing the subject or the technician to move a body part of the subject so as to cause inertial sensors to undergo motion from which the relative locations and identities of the electrode assemblies 10 are determined. In situations where the location identifying elements include mechanical switches, the subject or technician is provided with prompts instructing either one of them to press the switches in some specified order, from which the relative locations of the electrode assemblies 10 can be inferred. In the case of using optical sensors, the controller 30 can instruct the subject or technician to aim a light source (e.g., a laser pen / pointer) at the various electrode assemblies 10 in some specified order, with the optical sensors then transmitting signals in response to detecting the light.

In some embodiments, determining which output port of the switch array 40 is connected to which electrode assembly (whose location relative to other electrode assemblies has been determined) can be accomplished by sequentially sending a signal through each output port of the switch array 40, and receiving reply signals from the respective electrode assemblies 10 connected to those ports. In the embodiments depicted in FIG. 2, determination of locations of the electrode assemblies, and possibly determination of connectivity of the electrode assemblies 10 to the output ports can be performed using a machine learning system to detect electrode assemblies, cables, and possibly output ports appearing in a captured image of a scene of the system 200.

After determining the relative locations of the various deployed electrode assemblies and their connectivity to the output ports of the switch array 40, the controller 30 can dynamically configure the AC signal generator 20 and/or the switch array 40 to establish electrical paths, and to apply AC voltages, to the various electrode assemblies 10. For example, if the TTFields therapy to be applied to the subject requires repeated impositions of electrical fields between the front-and-back electrode assemblies 10D and 10B, followed by imposition of electrical fields between side electrode assemblies 10A and 10C, the controller will cause, at a first time interval, an electrical path between the output of the AC signal generator 20 to output ports #2 and #3 of the switch array 40 to be created, and to then cause AC voltages to be applied at those ports (for some therapeutically needed time period and voltage level). Then, at a second time interval, the controller will cause electrical paths between the AC signal generator's output and ports 1 and 4 of the switch array 40 to be established, and cause AC voltages to be applied at those output ports for a therapeutically determined time period and voltage level. The controller can then repeat this process of creating different electrical paths and applying voltages for a therapeutically desired time interval.

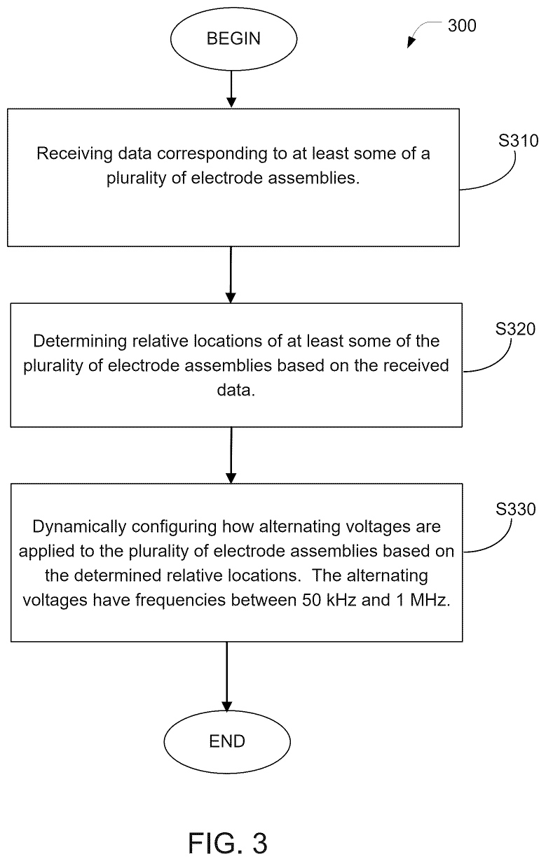

FIG. 3 is a flowchart of an example procedure 300 for using a plurality of electrode assemblies positioned on or in a subject's body to apply alternating electric fields to a region of interest within the subject's body. The procedure 300 includes receiving (S310) data corresponding to at least some of the plurality of electrode assemblies (such as the assemblies 10 shown in FIGS. 1 and 2), determining (S320) relative locations of at least some of the plurality of electrode assemblies based on the received data, and dynamically configuring (S330) how alternating voltages are applied to pairs of the plurality of electrode assemblies based on the determined relative locations. The alternating voltages have frequencies between 50 kHz and 1 MHz.

The procedure 300 can be implemented according to various embodiments, some of which were described in relation to FIGS. 1 and 2. Further details of the different embodiments in which the operations of the procedure 300 can be performed are provided below. In some embodiments, the dynamically configuring (as shown in S330) comprises (a) recognizing which of the plurality of electrode assemblies are disposed on opposite sides of the region of interest based on the determined relative locations, and (b) grouping the electrode assemblies into pairs so that the alternating voltages will be applied to electrode assemblies that are disposed on opposite sides of the region of interest.

In some embodiments, each of the plurality of electrode assemblies can be connected to a respective one of a plurality of output terminals of a switch array. In such embodiments, the dynamically configuring can include dynamically configuring electrical connections between output terminals of an AC signal generator and the output terminals of the switch array.

In some embodiments, the determining of relative locations (as shown in S320 of FIG. 3) can include determining relative locations for a subset of the plurality of electrode assemblies that does not include all of the electrode assemblies. In these embodiments, the location(s) of the remaining electrode assemblie(s) that were not specifically determined can be inferred from the determined locations of the subset of the plurality of electrode assemblies. For example, if the system has determined that three of the electrode assemblies are positioned on the front, left, and right sides of the subject's head, respectively, it can sometimes be appropriate to assume that the fourth electrode assembly is positioned on the back of the subject's head.

In some embodiments, the receiving of data (shown in S310 of FIG. 3) can include receiving at least one signal from each of a plurality of location identifying elements 12, with each of the plurality of location identifying elements being associated with a given one of the plurality of electrode assemblies 10 positioned on the subject's body. In such embodiments, determining of relative locations can be based on the at least one signal received from each of the plurality of location identifying elements 12. Optionally, in these embodiments, the receiving of data can occur in response to at least one issued prompt, and the method further comprises issuing the at least one prompt.

In some embodiments, each of the location identifying elements 12 can include an inertial sensor. Each of the inertial sensors can include at least one of an accelerometer, a gyroscope, and/or a magnetometer. In various embodiments, each of the location identifying elements 12 can include an optical sensor.

In some embodiments, each of the location identifying elements 12 can include a switch that is actuatable by the subject. In such embodiments, the receiving of data can include receiving an indication that each of the switches has been actuated by the subject at a respective time after a corresponding prompt has been issued to the subject.

In some embodiments, the receiving of data (as shown in S310 of FIG. 3) can include receiving captured image data of the plurality of electrode assemblies positioned on the subject's body. In such embodiments, the determining of relative locations can include (a) identifying, using a trained machine learning system implementing an electrode assembly recognition model, the at least some of the plurality of electrode assemblies included in the image data, and (b) determining relative locations for the at least some of the plurality of electrode assemblies based on the identified electrode assemblies included in the image data. Optionally, QR codes or bar codes may be provided on some or all of the components in the system, in which case the image data will include images of the QR codes or bar codes. This can help the system recognize the various components fully automatically, without requiring the user to perform any activities like moving their head or pressing buttons.

Thus, and as also discussed in relation to FIG. 2, an image capturing device (such as the camera 60 of FIG. 2) acquires an image of a scene that includes the subject and a view of at least some of the electrode assemblies positioned on the subject's body (including QR codes or bar codes, when those are present in the image). The acquired image is transferred to a machine learning (ML) system (that may be implemented by the controller 30 or in a remote server) which has been trained (prior to commencing use of the ML system, and optionally intermittently during runtime) to identify electrode assemblies, and other features in the scene such as electric cables, body parts, etc.

The initial training of the ML system can rely on processing images of electrode assemblies deployed on subjects'bodies, with the electrode assemblies having been labeled (by reviewers of the images in the training set) to define the ground truth for the training set. During the training stage, parameters of the ML system (e.g., the weights of connections between nodes in different layers of the ML system) can be optimized to minimize the error between the ground truth and the output of the ML system.

During runtime the identified elements from the scene are used to determine the relative locations and identify the electrode assemblies appearing in the acquired image. Electrode assemblies not appearing in a capture image of the scene can have their relative location inferred, as more particularly discussed above. As noted, a calibration/configuration process that uses location identifying elements (such as the elements 12 shown in FIGS. 1 and 2) to identify electrode assemblies and determine their relative locations can be used in addition to determining relative location using an ML system (e.g., to independently confirm the accuracy of the determined relative locations), or if the ML system failed to identify the relative locations of the electrode assemblies.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A method for using a plurality of electrode assemblies positioned on or in a subject's body to apply alternating electric fields to a region of interest within the subject's body, the method comprising:
receiving data corresponding to at least some of the plurality of electrode assemblies;
determining relative locations of at least some of the plurality of electrode assemblies based on the received data; and
dynamically configuring how alternating voltages are applied to the plurality of electrode assemblies based on the determined relative locations, wherein the alternating voltages have frequencies between 50 kHz and 1 MHz.

2. The method of claim 1, wherein the dynamically configuring comprises recognizing which of the plurality of electrode assemblies are disposed on opposite sides of the region of interest based on the determined relative locations, and grouping the electrode assemblies into pairs so that the alternating voltages will be applied to electrode assemblies that are disposed on opposite sides of the region of interest.

3. The method of claim 1, wherein the dynamically configuring comprises recognizing which of the plurality of electrode assemblies are disposed on opposite sides of the region of interest based on the determined relative locations, and grouping the electrode assemblies into groups so that the alternating voltages will be applied to electrode assemblies that are disposed on opposite sides of the region of interest.

4. The method of claim 1, wherein each of the plurality of electrode assemblies is connected to a respective one of a plurality of output terminals of a switch array, and wherein the dynamically configuring comprises dynamically configuring electrical connections between output terminals of an AC signal generator and the output terminals of the switch array.

5. The method of claim 1, wherein the determining of relative locations comprises determining relative locations for a subset of the plurality of electrode assemblies comprising fewer electrode assemblies than a total number of the plurality of electrode assemblies.

6. The method of claim 1, wherein the receiving of data comprises receiving at least one signal from each of a plurality of location identifying elements, wherein each of the plurality of location identifying elements is associated with a given one of the plurality of electrode assemblies positioned on the subject's body, and wherein the determining of relative locations is based on the at least one signal received from the each of the plurality of location identifying elements.

7. The method of claim 6, wherein the receiving of data occurs in response to at least one issued prompt, and wherein the method further comprises issuing the at least one prompt.

8. The method of claim 6, wherein each of the location identifying elements comprises an inertial sensor.

9. The method of claim 6, wherein each of the location identifying elements comprises an optical sensor.

10. The method of claim 6, wherein each of the location identifying elements comprises a switch that is actuatable by the subject.

11. The method of claim 1, wherein the receiving of data comprises receiving captured image data of the plurality of electrode assemblies positioned on the subject's body, and wherein the determining of relative locations comprises (a) identifying, using a trained machine learning system implementing an electrode assembly recognition model, the at least some of the plurality of electrode assemblies included in the image data, and (b) determining relative locations for the at least some of the plurality of electrode assemblies based on the identified electrode assemblies included in the image data.

12. An apparatus for applying alternating electric fields to a region of interest within a subject's body, the apparatus comprising:
a controller;
an AC signal generator that generates an output at a frequency between 50 kHz and 1 MHz;
and a switch array electrically coupled to the AC signal generator, wherein the switch array is dynamically configurable to route the output of the AC signal generator to selected ones of a plurality of electrode assemblies based on at least one control signal that arrives from the controller, wherein the controller is configured to select which of the plurality of electrode assemblies will receive the output of the AC signal generator at respective times based on signals that arrive from a plurality of location identifying elements, each of which is associated with a given one of the plurality of electrode assemblies.

13. The apparatus of claim 12, wherein the controller is configured to (a) recognize which of the plurality of electrode assemblies are disposed on opposite sides of the region of interest based on the signals that arrive from the plurality of location identifying elements, and (b) group the electrode assemblies into pairs so that the output of the AC signal generator is routed to electrode assemblies that are disposed on opposite sides of the region of interest.

14. The apparatus of claim 12, wherein the controller is configured to (a) recognize which of the plurality of electrode assemblies are disposed on opposite sides of the region of interest based on the signals that arrive from the plurality of location identifying elements, and (b) group the electrode assemblies into groups so that the output of the AC signal generator is routed to electrode assemblies that are disposed on opposite sides of the region of interest.

15. The apparatus of claim 12, wherein the controller is configured to cause, based on the signals that arrive from the plurality of location identifying elements, actuation of one or more switches of the switch array to electrically connect an output of the AC generator to pairs of output ports of the switch array.

16. The apparatus of claim 15, wherein the controller is further configured to cause, based on the signals that arrive from the plurality of location identifying elements and based on a schedule specifying the timing at which different pairs of electrode assemblies should be activated or de-activated, actuation of one or more switches of the switch array to electrically connect the output of the AC generator to the pairs of output ports of the switch array.

17. The apparatus of claim 12, further comprising:
the plurality of electrode assemblies; and
the plurality of location identifying elements.

18. The apparatus of claim 17, wherein each of the electrode assemblies is configured to adhere to the subject's body, and wherein each of the location identifying elements is configured to produce at least one signal representative of a relative location of the associated given one of the plurality of electrode assemblies, and to communicate the at least one signal to the controller.

19. An electrode assembly for applying alternating electric fields to a region of interest within a subject's body, the electrode assembly comprising:
one or more conductive pads having a front surface, wherein a collective area of the one or more conductive pads is at least 5 cm²;
a layer of conductive adhesive disposed on the front surface of the one or more conductive pads;
an inertial sensor configured to generate an output; and
a cable configured to route a signal from an AC signal generator to the one or more conductive pads.

20. The electrode assembly of claim 19, wherein the cable is further configured to route the output of the inertial sensor to a remote controller.
